(19) United States
(12) Patent Application Publication
(10) Pub. No.: US 2008/0221528 A1
(43) Pub. Date: Sep. 11, 2008

(54) NEEDLE PROTECTING DEVICE COMPRISING AN INTEGRATED NEEDLE CARRIER REMOVING DEVICE FOR REMOVING A NEEDLE CARRIER FROM AN INJECTION APPARATUS

(76) Inventor: Marc Lanz, Lobsigen (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/037,570

(22) Filed: Feb. 26, 2008

(30) Foreign Application Priority Data
Feb. 27, 2007 (DE) DE 102007009340.5

(51) Int. Cl. A61M 5/32

(52) U.S. Cl. 604/192

(57) ABSTRACT

A needle protecting device for use with an injection apparatus, wherein the injection apparatus has an associable needle carrier, the needle protecting device including a needle protecting sleeve and an integrated needle carrier removing device for removing the needle carrier from the injection apparatus.

### CROSS-REFERENCE TO RELATED APPLICATIONS

This application claims priority to German Application No. DE 10 2007 009 340.5 filed on Feb. 27, 2007, the contents of which are incorporated in their entirety herein by reference.

### BACKGROUND

The present invention relates to devices or apparatus for delivering, injecting, infusing, dispensing or administering a substance, and to methods of making and using such devices. More particularly, it relates to a needle covering and/or protecting device comprising an integrated needle carrier removing device for an injection apparatus, for removing a needle carrier from an injection apparatus. The needle protecting device comprising the integrated needle carrier removing device is detachably fastened to the injection apparatus. The injection apparatus may be a simple syringe or an injection pen, such as is used for treating diabetes and also, more recently, in other treatments.

When handling injection apparatus, there is a risk of patients or medical staff being injured by pointed injection needles and infected by injection needles which have already been used. Needle protecting devices with a movable needle protector, shield, cover or other protection structure have been developed to prevent this. Typically, the needle protection structure can be moved back and forth once in the longitudinal direction of the injection needle and, after use, can be automatically blocked or locked in a distal protective position in which the injection needle is inaccessible, such that it no longer moves in the proximal direction and such that the injection needle or at least the tip thereof cannot be exposed. Such needle protecting devices are, for example, known from WO 01/91837 A and U.S. Pat. No. 6,773,415 B2.

After using an injection needle, which can be fixedly bonded or fused to a needle carrier and protrudes or extends out of the needle carrier, it is desirable to remove the needle carrier together with the injection needle from an injection apparatus and replace it with a new one. When removing the needle carrier bearing an injection needle, it is necessary to ensure that patients or medical staff do not prick themselves with the injection needle and that the needle carrier can be disposed of as easily and safely as possible in a disposal container. A needle carrier, needle and a needle carrier removing device is known from DE 10255134 A1. A disadvantage of the known needle carrier removing device is that removing the needle carrier comprising the injection needle poses a certain risk of injury, since after it has been used, the injection needle is exposed and the injection apparatus comprising the needle carrier and the exposed injection needle have to be guided into a separately provided needle carrier removing device to remove the needle carrier.

### SUMMARY

An object of the present invention is to provide a needle protecting device comprising an integrated needle carrier removing device for removing a needle carrier from an injection apparatus, which increases the security against pricking injuries and makes it easier to pull off or remove a needle carrier from an injection apparatus.

In one embodiment, the present invention comprises a needle protecting device for an injection apparatus comprising a needle carrier comprising a needle protecting sleeve and an integrated needle carrier removing device for removing the needle carrier from the injection apparatus.

In one embodiment, the present invention comprises an injection device, a needle carrier associable with the injection device, and a needle cover and/or protector comprising an integrated needle carrier remover for removing the needle carrier from the injection apparatus.

In one embodiment, the present invention comprises a needle carrier for use with an injection device, the needle carrier comprising a needle covering sleeve and a needle carrier remover for removing the needle carrier from an injection device.

In the following, the distal position of the needle protecting device comprising the integrated needle carrier removing device refers to a protective position or the position in which the injection needle is covered or substantially covered by the needle protecting device or a portion thereof. The proximal position of the needle protecting device is the position in which the injection needle or a portion thereof is exposed and extends or protrudes out of the needle protection.

In one embodiment, a needle protecting device comprising an integrated needle carrier removing device is fitted onto an injection apparatus during use, for example by a clamping connection, screw connection, snap-fit connection, bayonet connection, engaging connection or other fastening connection. The injection apparatus comprises a detachably connected needle carrier comprising an injection needle which is fixedly attached, for example fused or bonded, to it. In accordance with the invention, a clamping element, screw element, snap-fit element, bayonet element, guide element or other fastening element is formed on a casing portion of the injection apparatus, and complementary or counter clamping element, screw element, snap-fit element, bayonet element, guide element or other fastening counter element is formed on a casing portion of the needle protecting device. The fastening and/or fastening counter elements can be a fixed part or a separate part of the injection apparatus or the needle protecting device. Once the connection between the fastening element and the fastening counter element has been established, the injection apparatus is connected to the needle protecting device such that it is secured against shifting axially and against rotating.

In one embodiment, the needle protecting device, comprising the integrated needle carrier removing device, in accordance with the present invention comprises a sleeve-shaped base body and a needle protecting sleeve. An injection needle of a needle carrier detachably fastened to an injection apparatus protrudes beyond a distal front end of the base body. The needle protecting sleeve is mounted on the base body such that it can axially shift and, in some preferred embodiment, such that it can rotate about its longitudinal axis. In a distal initial protective position, the needle protecting sleeve surrounds the injection needle. In this protective position, in some preferred embodiments, the needle protecting sleeve is secured or locked by a lock against inadvertently shifting or exposing the injection needle. Once the lock is released, the needle protecting sleeve can be shifted into a proximal rear position. For the injection, the injection apparatus fitted with the needle protecting sleeve is pushed against the body using the needle protecting sleeve. Due to the external force, the injection apparatus moves in the distal direction relative to the needle protecting sleeve, such that the injection needle pierces the body. The needle protecting sleeve correspondingly completes a movement relative to the injection apparatus, in the proximal direction, as far as a releasing position in which the injection portion of the injection needle penetrating the body protrudes beyond the needle protecting sleeve in the distal direction. When the injection needle is drawn out of the body, the needle protecting sleeve moves back in the distal direction, due to being charged with an elasticity force of a spring member, as far as a protective position in which it extends beyond the injection needle including the needle tip. Once the protective position has been reached, movement of the needle protecting sleeve is blocked or prevented by its being latched by the lock, such that it cannot be moved back in the proximal direction again, relative to the injection needle. The latching by the lock can be triggered actively by the user or automatically by the injection apparatus.

In some embodiments, in the protective position and after the injection has been performed, the needle protecting sleeve is locked against sliding back relative to the base body and the injection needle. This locking can be achieved using various locking devices, for example by a first and second latching element. One of the latching elements can be a sleeve-shaped latching ring which can comprise at least one protrusion which protrudes radially inwardly, for example a collar, on its inner surface area and a protrusion which protrudes radially outwardly, for example a cam, on its outer surface area. With respect to the sleeve-shaped latching ring, the collar is arranged substantially diametrically with respect to the cam. The other latching element can be formed by a circumferential annular groove or recess and can be provided on the outer surface area of the needle protecting sleeve. The collar of the first latching element can be slid into the groove of the second latching element due to an external force acting on the latching ring, until at least a part of the collar protrudes into the groove. As soon as at least a part of the collar protrudes into the groove, a latching engagement is established and the needle protecting sleeve cannot be moved out of the protective position, in which the injection needle is covered by the needle protecting sleeve, into the proximal position in which the injection needle protrudes out of the needle protecting sleeve.

In some embodiments, the protective position can also be completed by a first and second locking means, wherein these locking means engage with each other in a locking engagement by the two locking means abutting axially against each other. One of the locking means can be connected to the needle protecting sleeve, secured against shifting, and the other locking means can be connected to the base body, secured against shifting. The locking engagement therefore prevents the needle protecting sleeve from shifting out of the protective position into the proximal position.

In some embodiments, a protective position of the needle protecting sleeve may be realized by another suitable locking mechanism, e.g., a cam and a guide rail for the cam or a catch element and a catch counter element.

After latching, the needle carrier comprising the injection needle in accordance with the present invention is to be safely removed from the injection apparatus. The needle carrier can be taken off, twisted off or screwed off the injection apparatus by various releasing devices, e.g., means comprising a first and second element which form a positive-fit engagement. One of the elements can be attached or attachable to a sleeve, for example a clamping element, wherein the sleeve and/or the clamping element at least partially encompasses the needle carrier. The clamping element can co-operate with the needle carrier in a non-positive fit. The other element can be fixedly connected or connectable to the needle protecting device, e.g. to the needle protecting sleeve. Due to a relative rotation between the needle protecting device and the injection apparatus about its longitudinal axis, or between the needle protecting sleeve and the base body about their longitudinal axes, the rotational force acts on the positive-fit engagement, such that the rotational force is transmitted from the needle protecting device, in particular the needle protecting sleeve, onto the needle carrier and the needle carrier can be released from the injection apparatus, wherein the needle carrier remains in the needle protecting device, without the risk of pricking injuries.

In some embodiments, the positive-fit engagement exists, for example, between at least one longitudinal web on the clamping element which extends parallel to the longitudinal axis of the needle carrier, and at least one complementarily formed recess on the needle protecting sleeve. Alternatively, the complementarily formed recess can be situated on the needle carrier and the longitudinal web can be on the needle protecting sleeve, wherein the longitudinal web extends parallel to the longitudinal axis of the needle protecting sleeve.

In some embodiments, the needle carrier can also be removed from, taken off, twisted off or screwed off the injection apparatus by a gripping member for gripping the needle carrier to be removed. To this end, the needle protecting device comprises the gripping member. This gripping member can be formed such that the gripping member at lest partially encompasses the needle carrier. To this end, the gripping member may have an elastically formed wall or other elastically flexible structure which can fix the needle carrier by a frictional-fit connection. Alternatively, a snap-fit structure can be provided. When the snap-fit mechanism is triggered, an arm of the needle protecting device comprising the needle carrier removing device then snaps into the space occupied by the needle carrier and fixes the needle carrier.

Once the needle carrier has been removed from the injection apparatus, the needle carrier is situated in the needle protecting device comprising the integrated needle carrier removing device. The removed needle carrier, which carries a used injection needle, wherein the blocked or locked needle protecting sleeve protrudes beyond the needle tip, can thus be safely disposed of together with the needle protecting device comprising the integrated needle carrier removing device.

In some embodiments, the needle carrier can be safely disposed of together with the used injection needle by providing the needle protecting device with a needle carrier ejecting means. Activating the needle carrier ejecting means of the needle protecting device releases the connection between the needle carrier and the clamping element, gripping member, snap-fit device or other holding member of the needle protecting device, such that the needle carrier can be disposed of in a secure container and the needle protecting device can be used again by connecting or reconnecting the needle protecting device to the injection apparatus again, e.g., by sliding, wherein the injection apparatus comprises a new needle carrier with a new injection needle. Alternatively, a separate needle carrier ejecting device can be provided, for example a pin-shaped needle carrier ejector. In this instance, to remove the needle carrier comprising the used injection needle from the needle protecting device, the needle carrier ejector is slid through the open distal end of the needle protecting sleeve in the axial direction against the proximal end of the needle protecting sleeve and ejects the needle carrier comprising the used injection needle from the clamping element, gripping member, snap-fit device or other holding member, such that the needle carrier comprising the injection needle drops into the sleeve-shaped base body. The ejecting force acting on the needle carrier in the axial direction is greater than the clamping force or frictional-fit force acting between the clamping element, gripping member or other holding member and the needle carrier. The released needle carrier comprising the used injection needle can then be safely disposed of in a separate needle carrier disposal container.

### BRIEF DESCRIPTION OF THE DRAWINGS

FIG. 1 is an exploded view of an exemplary embodiment of a needle protecting device comprising an integrated needle carrier removing device;

FIG. 2a depicts one embodiment of a needle protecting device comprising an integrated needle carrier removing device, in which a longitudinal section A-A is indicated;

FIG. 2b is a longitudinal section along line A-A of FIG. 2a, wherein the needle protecting device is in a distal position;

FIG. 3a is a representation of the needle protecting device, in which a longitudinal section B-B is indicated;

FIG. 3b is a longitudinal section along line B-B of FIG. 3a;

FIG. 4a is a representation of the needle protecting device, in which a longitudinal section C-C is indicated;

FIG. 4b is a longitudinal section along line C-C of FIG. 4a, wherein the needle protecting device is in a proximal position;

FIG. 5a is a representation of the needle protecting device, in which a longitudinal section D-D is indicated;

FIG. 5b is a longitudinal section along line D-D wherein the needle protecting device is in a proximal position;

FIG. 6 is a perspective representation of a latching ring;

FIG. 7, including FIGS. 7a-7d, depicts an embodiment of an injection sequence using a needle protecting device comprising an integrated needle carrier removing device.

### DETAILED DESCRIPTION

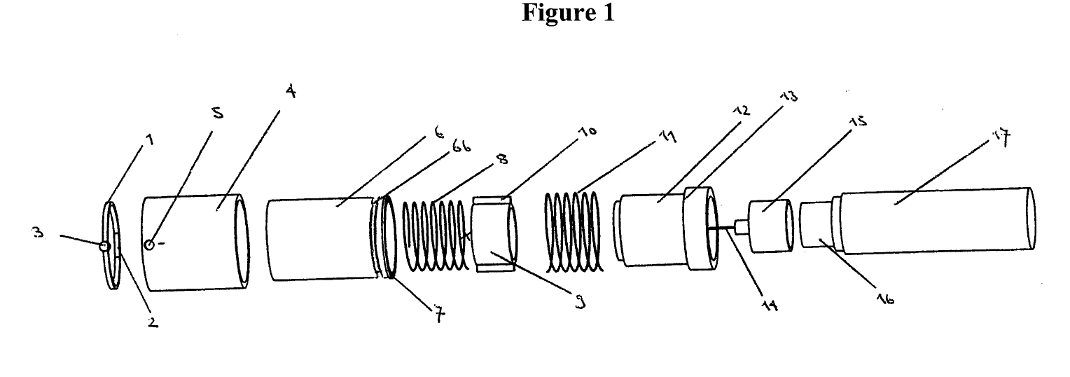

FIG. 1 is an exploded representation of one embodiment of a needle protecting device comprising an integrated needle carrier removing device, which can be attached, plugged, clamped, snap-fitted or screwed onto a distal end of an injection apparatus 17, wherein the injection apparatus 17 bears a detachable needle carrier 15 comprising a fixedly fused or bonded injection needle 14. The needle protecting device comprising the integrated needle carrier removing device is fitted onto the injection apparatus 17 in such a way that the injection needle 14 is enclosed by the needle protecting device prior to the injection, protrudes out of the needle protecting device during the injection, and is again enclosed by the needle protecting device after the injection.

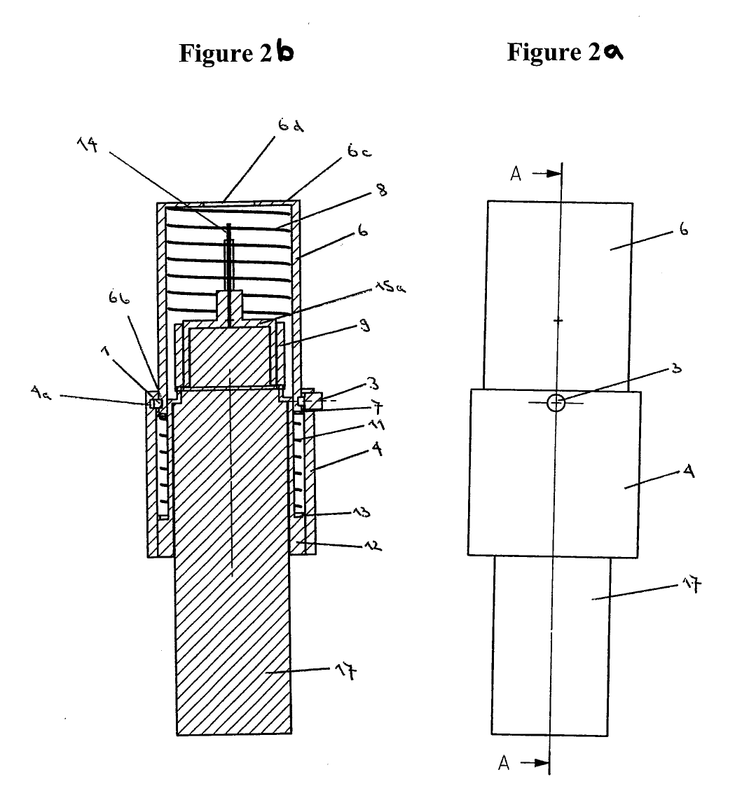

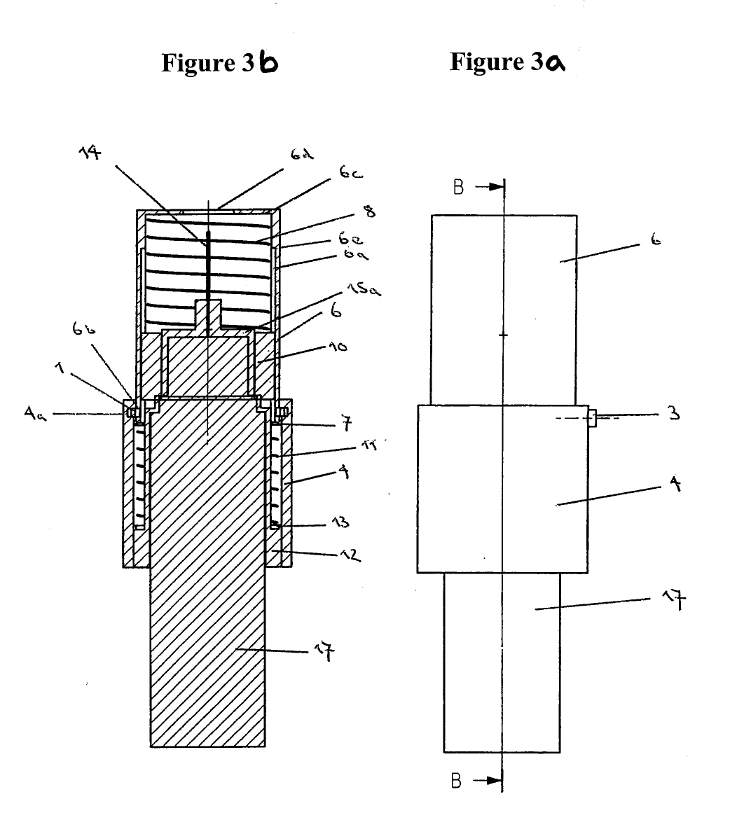

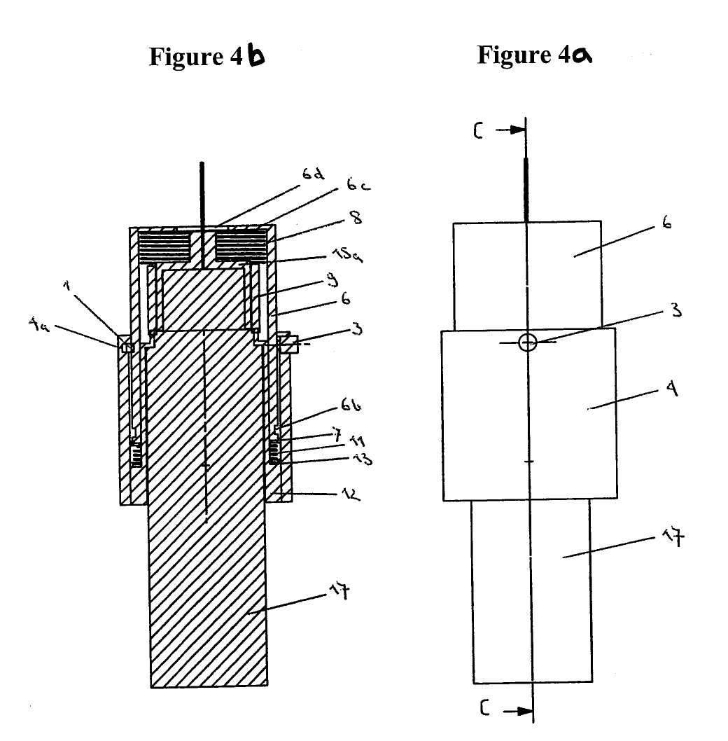

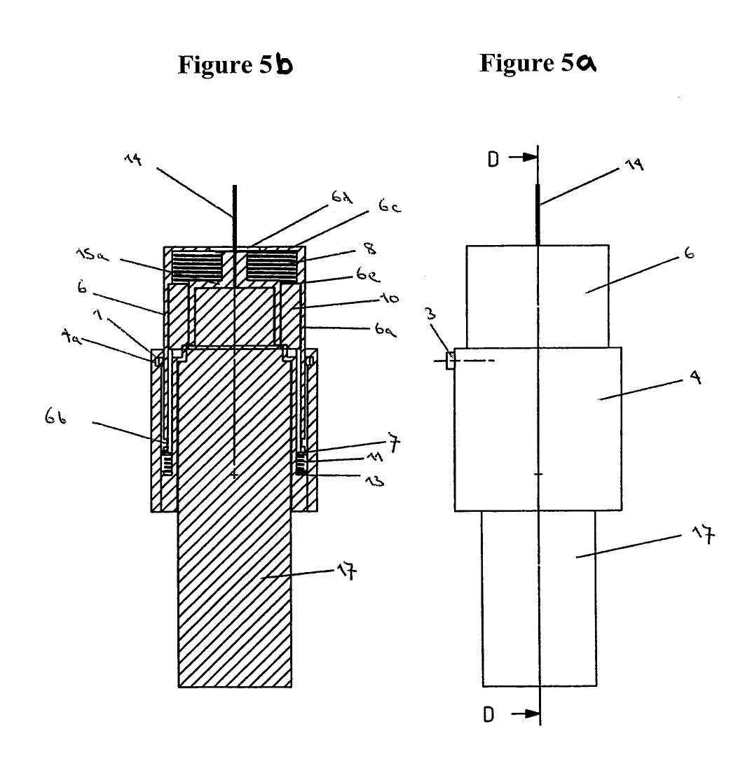

The device in accordance with the present invention comprises a latching ring 1, an outer casing 4, a needle protecting sleeve 6, a first spring member 8, a clamping element 9, a second spring member 11 and an inner casing 12. The latching ring 1 has a cam 3, which protrudes radially outwardly, on its outer surface side and a collar 2, which protrudes radially inwardly, on its inner side. The hollow cylindrical outer casing 4 comprises an opening 5 at the distal end of its surface area, which is dimensioned such that the cam 3 of the latching ring 1 can protrude into the opening 5. The inner surface area of the outer casing 4 comprises an annular groove 4a in the circumferential direction (cf. FIG. 2b, FIG. 3b, FIG. 4b, FIG. 5b), wherein the opening 5 lies in the groove 4a. The groove 4a runs approximately parallel to the transverse axis of the outer casing 4. The proximal end of the outer surface area of the cylindrical needle protecting sleeve 6 has a circumferential stop 7 which protrudes radially outwardly and is approximately parallel to the transverse axis of the needle protecting sleeve. A groove 6b which is annular in the circumferential direction is also provided on the outer surface area of the needle protecting sleeve 6. The groove 6b of the needle protecting sleeve 6 is offset in the distal direction relative to the stop 7 of the needle protecting sleeve 6 and runs or extends approximately parallel to the stop 7 of the needle protecting sleeve 6. The needle protecting sleeve 6 has a guide groove 6a on its inner surface area (cf. FIG. 3b, FIG. 5b). The guide groove 6a runs parallel to the longitudinal axis of the needle protecting sleeve 6. The distal end of the guide groove 6a is bounded by a stop 6e (cf. FIG. 3b, FIG. 5b). The needle protecting sleeve 6 is closed by a terminal wall 6c at the distal end, wherein the terminal wall 6c (cf. FIG. 2b, FIG. 3b, FIG. 4b, FIG. 5b) comprises an opening 6d (cf. FIG. 2b, FIG. 3b, FIG. 4b, FIG. 5b) which is formed such that the injection needle 14 can be guided through the opening 6d of the terminal wall 6c. The first spring member 8 is axially supported on the terminal wall 6c of the needle protecting sleeve 6 on one side and on a terminal wall 15a of the needle carrier 15 and on the clamping element 9 on the other side. The first spring member 8 exerts on the clamping element 9 an elasticity force acting in the proximal direction. The outer surface area of the sleeve-shaped clamping element 9 comprises a protrusion 10 which protrudes radially outwardly and extends along the longitudinal axis of the clamping element 9. The second spring member 11 is axially supported on the stop 7 of the needle protecting sleeve 6 and on a stop 13 of an inner casing 12. The second spring member 11 exerts on the needle protecting sleeve 6 an elasticity force acting in the distal direction. The circumferential stop 13, which protrudes radially outwardly, is attached at the proximal end of the outer surface area of the inner casing 12. The inner surface area of the inner casing 12 is formed such that the needle protecting device comprising the integrated needle carrier removing device can be connected to the injection apparatus 17 in a clamping connection, screw connection, snap-fit connection, bayonet connection, engaging connection or other fastening or mounting connection. The distal end of the injection apparatus 17 is detachably connected or coupled to the needle carrier 15, for example by a rotational connection. An injection needle 14 protrudes out of or from the needle carrier 15. The injection needle 14 is fixedly connected, such as for example fused or bonded, to the needle carrier 15.

FIG. 2b shows a longitudinal section of the needle protecting device comprising the integrated needle carrier removing device in its blocked distal position, wherein the needle protecting sleeve 6 protrudes beyond the injection needle 14, and the needle protecting sleeve 6 is blocked or locked and cannot be shifted into the proximal rear position. Next to this longitudinal section, FIG. 2a shows an overall representation of the needle protecting device comprising the integrated needle carrier removing device, in which the position of the longitudinal section A-A is indicated.

In the depicted embodiment, the inner casing 12 and the outer casing 4 are each generally cylindrical and sleeve-shaped, wherein the outer surface area of the inner casing 12 is connected to the inner surface area of the outer casing 4, secured against shifting axially and against rotating. The inner casing and the outer casing can also consist of a prefabricated part. An annular gap, in which the second spring member 11 is mounted, is between the inner casing 12 and the outer casing 4. The annular gap extends approximately parallel to the longitudinal axis of the inner casing 12 and the outer casing 4. The second spring member 11 is axially supported on the stop 13 of the inner casing 12 and on the stop of the needle protecting sleeve 6, such that the second spring member exerts on the needle protecting sleeve 6 an elasticity force which acts in the distal direction and is large enough that the needle protecting sleeve 6 is slid over the injection needle 14. The outer surface area of the inner casing 12 forms a guide for the needle protecting sleeve 6—in the exemplary embodiment, a sliding guide. The annular groove 6b, which can accommodate the sleeve-shaped latching ring 1, is generally at the proximal end of the needle protecting sleeve 6.

A third spring member, which cannot be seen in FIG. 2b, is mounted in the groove 4a of the outer casing 4 and exerts on the latching ring 1 an elasticity force which acts in the transverse direction, such that the latching ring 1 is charged or urged in the transverse direction to the longitudinal axis. The collar 2, which protrudes radially inwardly on the inner surface side of the latching ring 1, protrudes into the groove 6b of the needle protecting sleeve 6 and is in a latching engagement with the groove 6b of the needle protecting sleeve 6. The needle protecting sleeve 6 cannot be shifted in the proximal direction. The cam 3, which protrudes radially outwardly on the outer surface area of the latching ring 1, protrudes through the opening 5 of the outer casing 4.

The clamping element 9 is hollow, cylindrical and surrounds the needle carrier 15 which is fitted on the injection apparatus 17. The clamping element 9 is arranged such that it can move axially in the needle protecting sleeve 6 and is axially supported by the first spring member 8 and the distal terminal end of the inner sleeve 12. A stop can additionally be attached at the proximal end of the needle protecting sleeve 6 to support the clamping element 9 on the inner surface side. The clamping element 9 is formed such that, when the needle protecting device is fitted onto an injection apparatus 17, the inner surface area of the clamping element 9 can be slid over the outer surface area of the needle carrier 15 due to an elasticity force of the first spring member 8 which acts on the clamping element 9 in the proximal direction. The clamping element 9 slides over the needle carrier 15 until a clamping fit or frictional fit is established between the clamping element 9 and the needle carrier 15.

FIG. 3b, like FIG. 2, shows the needle protecting device in an initial state. In FIG. 3b, however, the position of the longitudinal section has been rotated by 90 degrees relative to the longitudinal section in FIG. 2b. Next to this longitudinal section, FIG. 3a also shows an overall representation of the needle protecting device comprising the integrated needle removing device, in which the position of the longitudinal section B-B is indicated.

On its outer surface area, the clamping element 9 comprises at least one protrusion 10 which protrudes radially outwardly and extends along the longitudinal axis of the clamping element 9. The exemplary embodiment shows two protrusions 10 which are uniformly spaced apart from each other. On its inner surface area, the needle protecting sleeve 6 comprises two grooves 6a which run or extend parallel to its longitudinal axis and can accommodate the protrusions 10 in a positive fit. The protrusion 10 is mounted such that it can move axially in the groove 6a. When the needle protecting sleeve 6 is rotated about its longitudinal axis relative to the outer casing 4, which is connected to the inner casing 12, secured against rotating, the rotational force is transmitted onto the needle carrier 15, such that the needle carrier 15 can be twisted, drawn or screwed off the injection apparatus 17.

FIG. 4b shows a longitudinal section of the needle protecting device, in a proximal position, wherein the injection needle 14 protrudes beyond the needle protecting sleeve 6 in the distal direction. The needle protecting sleeve 6 is not blocked or locked and can be shifted or returned from the proximal rear position into the distal front position. Next to this longitudinal section, FIG. 4a shows an overall representation of the needle protecting device, in which the position of the longitudinal section C-C is indicated.

The user exerts on the cam 3, which protrudes radially outwardly on the outer surface area of the latching ring 1, a force which acts against the third spring member in the transverse direction, such that the latching ring 1 is shifted in the transverse direction. The collar 2, which protrudes radially inwardly on the inner surface side of the latching ring 1, is pressed into the groove 4a of the outer casing 4, such that the collar 2 no longer protrudes into the groove 6b of the needle protecting sleeve. The needle protecting sleeve 6 can be shifted into the proximal position by pressing the needle protecting sleeve 6 against the body. For the injection, the injection apparatus 17 comprising the detachably connected needle carrier 15, which is enclosed by the clamping element 9 in a clamping fit or frictional fit, is moved in the distal direction relative to the needle protecting sleeve due to the external force, such that the injection needle 14 protrudes out of the needle protecting sleeve 6.

FIG. 5b, like FIG. 4b, shows the needle protecting device in the released position. In FIG. 5b, however, the position of the longitudinal section has been rotated by 90 degrees relative to the longitudinal section in FIG. 4b. Next to this longitudinal section, FIG. 5a shows an overall representation of the needle protecting device comprising the integrated needle removing device, in which the position of the longitudinal section D-D is indicated.

The needle protecting sleeve 6 is guided by the outer surface area of the inner casing 12 into a proximal rear position, until the protrusion 10 of the clamping element 9, which protrudes radially outwardly, abuts the stop 6e of the needle protecting sleeve 6.

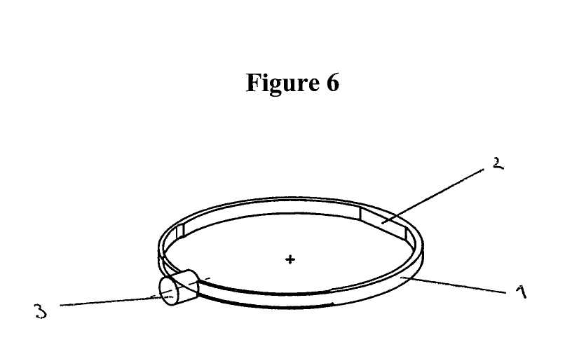

FIG. 6 shows the latching ring 1. The sleeve-shaped latching ring 1 has the cam 3, which protrudes radially outwardly, on its outer surface area and the collar 2, which protrudes radially inwardly, on the inner surface area. The third spring member exerts an elasticity force on the latching ring 1. The third spring member is axially supported in the groove 4a of the outer casing 4 and on the outer surface area of the collar 2 of the latching ring 1. The needle protecting device, comprising the integrated needle carrier removing device, in accordance with the present invention can also lack a latching ring 1 or other latching mechanism, such that the needle protecting sleeve 6 is unlatched before and after the needle carrier 15 is removed.

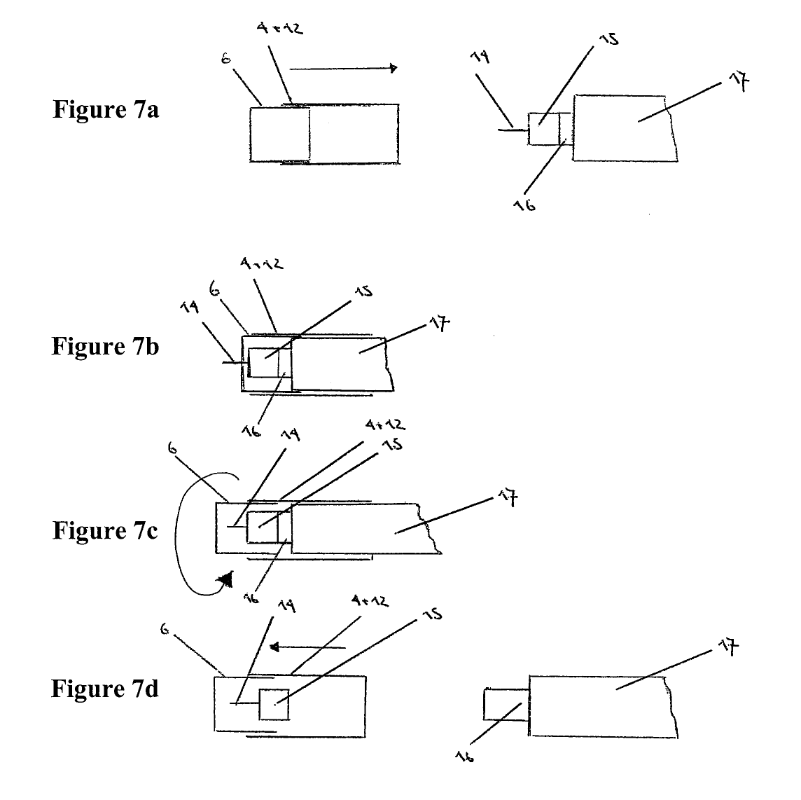

In the following, a sequence for using the needle protecting device comprising the integrated needle carrier removing device is described on the basis of FIG. 7, including FIGS. 7a to 7d.

As shown in FIG. 7a, the needle protecting device comprising the integrated needle carrier removing device is detachably fastened to a distal (or forward) end of an injection apparatus 17. When the needle protecting device comprising the integrated needle carrier removing device is fitted, the clamping element 9 is slid over the needle carrier 15 due to an elasticity force of the first spring member 8 acting on the clamping element 9 in a proximal (rearward) direction, such that the clamping element 9 encloses the needle carrier 15. Alternatively, the clamping element 9 can be connected to the inner casing 12 in a connection which is secured against shifting and against rotating, such that a first spring member 8 is not needed to slide the clamping element over the needle carrier 15. The clamping element 9 establishes a clamping connection or frictional-fit connection with the needle carrier 15. On its outer surface area, the clamping element 9 comprises at least one protrusion 10 which protrudes radially outwardly. The protrusion 10 protrudes radially outwardly into the groove 6a and can slide in it, wherein the groove 6a is situated on the inner surface area of the needle protection, parallel to the longitudinal axis. In the distal initial protective position, the needle protecting sleeve 6 surrounds or substantially surrounds the injection needle 14. In this protective position, the needle protecting sleeve 6 is secured against inadvertently shifting and exposing the injection needle by a latching engagement between the collar 2 of the latching ring 1, which protrudes radially inwardly, and the annular groove 6b on the outer surface area of the needle protection sleeve 6. The third spring member, which is axially supported in the groove 4a of the outer casing 4 and on the outer surface area of the collar 2 of the latching element 1, exerts on the latching ring 1 an elasticity force which acts in the transverse direction, such that the collar 2 of the latching ring 1 protrudes radially into the groove 6b of the needle protection 6. Once the latching engagement has been released, by the user exerting on the cam 3 of the latching ring 1 a force which acts against the third spring member in the transverse direction, the needle protecting sleeve 6 can be shifted into the proximal position, since the collar 2 of the latching ring 1 is shifted into the groove 4a of the outer casing 4. The injection needle 14 can penetrate the body by the injection apparatus 17 being moved in the distal direction relative to the needle protecting sleeve 6 due to the external force, such that the injection needle 14 pierces the body. The needle protecting sleeve 6 moves relative to the injection apparatus 17, into the proximal rear position. An injection can be performed by manually activating the injection device using, for example, a dosing button. An automatic injection can also be triggered by the needle protecting sleeve.

FIG. 7b shows the proximal position of the needle protecting device comprising the integrated needle carrier removing device. The needle protecting sleeve 6 is in a proximal position. The injection needle 14 protrudes or extends beyond the distal end of the needle protecting sleeve 6, such that the injection needle 14 penetrates the body and a product or substance can be injected into the body. The clamping element 9 encloses the needle carrier 15 and establishes a clamping connection or frictional-fit connection. The needle protecting sleeve 6 slides along the outer surface area of the inner casing 12, until the protrusion 10 of the clamping element 9 abuts the stop 6e of the needle protecting sleeve 6.

FIG. 7c shows the distal position of the needle protecting device. The injection needle 14 has been removed from the body and the needle protecting sleeve 6 thus moves back in the distal direction due to being charged with or urged by the elasticity force of the second spring member 11. Once the needle protecting sleeve 6 is in the protective position, the user no longer exerts a force on the cam 3 of the latching ring 1. The cam 3 protrudes through the opening 5 of the outer casing 4, since the cam 3 of the latching ring 1 is pressed into the opening 5 of the outer casing 4 due to the elasticity force of the third spring member. The collar 2 of the latching ring 1 protrudes into the groove 6b of the needle protecting sleeve 6 again and is in latching engagement with the groove 6b of the needle protecting sleeve 6. The latching engagement means that the needle protecting sleeve 6 cannot move back in the proximal direction relative to the injection needle 14 again. To release the needle carrier 15 from the injection apparatus 17, the needle protecting sleeve 6 is rotated about its longitudinal axis relative to the outer casing 4. The needle carrier 15 is twisted or screwed off the injection apparatus 17. The clamping element 9 is connected to the needle carrier 15 in a clamping or frictional fit, and the protrusion 10 of the clamping element 9 is connected to the needle protecting sleeve 6 in a positive fit in the circumferential direction. A relative rotation about the longitudinal axis between the needle protecting sleeve 6 and the outer casing 4 causes the rotational force to be transmitted from the needle protecting sleeve 6 onto the clamping element 9 and finally onto the needle carrier 15. The outer casing 4 is connected to the inner casing 12, secured against axially shifting and against rotating, and the inner casing 12 is connected to the injection apparatus 17 in a connection which is secured against axially shifting and against rotating.

FIG. 7d shows the removed needle carrier 15 comprising the injection needle 14, in the needle protecting device comprising the integrated needle carrier removing device. Once the needle carrier 15 comprising the injection needle 14 has been removed from, e.g., twisted or screwed off, the injection apparatus 17, the needle protecting device comprising the integrated needle carrier removing device can be taken off, twisted off, screwed off or drawn off the injection apparatus 17 by releasing the clamping connection, screw connection, snap-fit connection, bayonet connection, engaging connection or other fastening connection between the needle protecting device comprising the integrated needle carrier removing device and the injection apparatus 17. To remove the needle carrier 15 comprising the used injection needle 14 from the detached needle protecting device comprising the integrated needle carrier removing device, a pin-shaped needle carrier ejector is pushed in the axial direction through the opening 6d of the terminal wall 6c of the needle protecting sleeve 6, against the terminal wall 15a of the needle carrier 15. The ejecting force is greater than the clamping or frictional force between the clamping element 9 and the needle carrier 15, such that the needle carrier 15 drops into the inner casing 12 of the needle protecting device comprising the integrated needle carrier removing device. The detached needle carrier 15 comprising the used injection needle 14 can be disposed of in a safe and separate needle carrier disposal container, and the needle protecting device comprising the integrated needle carrier removing device is available for subsequent use.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising a needle carrier removeably connectable to the injection device and a needle cover comprising a needle carrier remover for removing the needle carrier from the injection apparatus.

2. A needle protecting device for an injection apparatus, comprising a needle carrier comprising a needle protecting sleeve and an integrated needle carrier removing device for removing the needle carrier from the injection apparatus.

3. The needle protecting device according to claim 2, wherein the needle carrier removing device comprises a gripping member which at least partially encompasses the needle carrier.

4. The needle protecting device according to claim 3, wherein the gripping member establishes at least one of a clamping-fit connection or frictional-fit connection with the needle carrier.

5. The needle protecting device according to claim 3, wherein the gripping member comprises at least one protrusion which protrudes radially outwardly.

6. The needle protecting device according to claim 5, wherein the needle protecting sleeve comprises a guide groove.

7. The needle protecting device according to claim 6, wherein the protrusion of the gripping member establishes a positive-fit connection with the guide groove of the needle protecting sleeve.

8. The needle protecting device according to claim 2, wherein the needle protecting sleeve is moveable against an elasticity force generated by a spring member from an initial position in which an injection needle is covered by the needle protecting sleeve to another position in which the injection needle protrudes out of the needle protecting sleeve, and wherein the sleeve is moveable from the another position toward the initial position.

9. The needle protecting device according to claim 8, wherein the sleeve is moveable from the another position toward the initial position into a protective position.

10. The needle protecting device according to claim 8, further comprising a first latching element and a second latching element that cooperate to provide a latching engagement that prevents the sleeve from moving toward the another position.

11. An injection apparatus comprising a removeable needle carrier and a removable needle protecting device comprising a needle protecting sleeve and an integrated needle carrier removing device for removing the needle carrier from the injection apparatus.

12. The injection apparatus according to claim 11, wherein the injection apparatus has a distal end, the distal end carrying the needle carrier, said needle carrier comprising an injection needle fixed thereto.
